(19) United States
(12) Patent Application Publication
(10) Pub. No.: US 2012/0244106 A1
(43) Pub. Date: Sep. 27, 2012

(54) USE OF THE COMBINATION OF TERIFLUNOMIDE AND GLATIRAMER ACETATE FOR TREATING MULTIPLE SCLEROSIS

(75) Inventors: William BYRNES, Cary, NC (US); Patrice DOUILLET, St. Gely du Fesc (FR); Gerald FRANGIN, Montpellier (FR)

(73) Assignee: SANOFI-AVENTIS U.S. LLC, Bridgewater, NJ (US)

(21) Appl. No.: 13/452,212

(22) Filed: Apr. 20, 2012

Related U.S. Application Data
(63) Continuation of application No. PCT/US2010/052423, filed on Oct. 13, 2010.
(60) Provisional application No. 61/261,954, filed on Nov. 17, 2009.
(60) Provisional application No. 61/286,153, filed on Dec. 14, 2009.

(30) Foreign Application Priority Data
Oct. 22, 2009 (EP) 09305999.6
Oct. 29, 2009 (EP) 09306036.6

(51) Int. Cl. A61K 31/785; A61P 25/00

(52) U.S. Cl. 424/78.37

(57) ABSTRACT

This invention is related to the use of the combination of teriflunomide or a pharmaceutically acceptable salt thereof and glatiramer acetate for treating multiple sclerosis.

This application is a Continuation of International Application No. PCT/US2010/052423, filed Oct. 13, 2010, which claims priority of European Application No. 09305999.6, filed Oct. 22, 2009, and European Application No. 09306036.6, filed Oct. 29, 2009, and claims benefit of U.S. Provisional Application No. 61/261,954 filed Nov. 17, 2009, and U.S. Provisional Application No. 61/286,153, filed Dec. 14, 2009, all of which are incorporated herein by reference in their entireties.

### FIELD OF THE INVENTION

This invention is related to the use of the combination of teriflunomide and glatiramer acetate for treating multiple sclerosis.

### BACKGROUND OF THE INVENTION

Multiple sclerosis (MS) is a debilitating, inflammatory, neurological illness characterized by demyelination of the central nervous system. The disease primarily affects young adults with a higher incidence in females. Symptoms of the disease include fatigue, numbness, tremor, tingling, dysesthesias, visual disturbances, dizziness, cognitive impairment, urologic dysfunction, decreased mobility, and depression. Four types classify the clinical patterns of the disease: relapsing-remitting, secondary progressive, primary-progressive and progressive-relapsing (S. L. Hauser and D. E. Goodkin, Multiple Sclerosis and Other Demyelinating Diseases in Harrison's Principles of Internal Medicine 14^{th} Edition, vol. 2, McGraw-Hill, 1998, pp. 2409-2419).

The exact etiology of MS is unknown; however, it is strongly suspected that the demyelination characteristic of the disease is the result of an autoimmune response perhaps triggered by an environmental insult, e.g. a viral infection. Specifically, it is hypothesized that MS is caused by a T-cell-mediated, autoimmune inflammatory reaction. The autoimmune basis is strongly supported by the fact that antibodies specific to myelin basic protein (MBP) have been found in the serum and cerebrospinal fluid of MS patients and these antibodies along with T-cells that are reactive to MBP and other myelin proteolipids increase with disease activity. Furthermore, at the cellular level it is speculated that T-cell proliferation and other cellular events, such as activation of B cells and macrophages and secretion of cytokines accompanied by a breakdown of the blood-brain barrier can cause destruction of myelin and oligodendrocytes. (R. A. Adams, M. V. Victor and A. H. Ropper eds, Principles of Neurology, McGraw-Hill, New York, 1997, pp. 903-921). Progressive MS (primary and secondary) may be based on a neurodegenerative process occurring with demyelination.

The use of (Z)-2-cyano-3-hydroxy-but-2-enoic acid-(4′-trifluoromethylphenyl)-amide (also known as teriflunomide, Formula I) for treating multiple sclerosis has been disclosed in U.S. Pat. No. 6,794,410. Although aforesaid patent discloses that teriflunomide may possibly be combined with another compound known to be effective for treating multiple sclerosis to treat the disease, however, no specific combination is disclosed to show such efficacy and safety in treating multiple sclerosis.

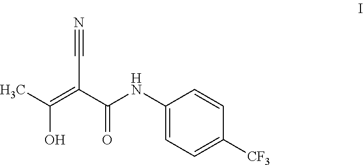

### SUMMARY OF THE INVENTION

This invention is related to a method for treating relapsing-remitting form of multiple sclerosis, in a patient in need thereof, comprising administering to the patient about 7 mg or about 14 mg of teriflunomide, and a pharmaceutically effective amount of glatiramer acetate.

### DETAILED DESCRIPTION OF THE INVENTION

As used above, and throughout the description of the invention, the following terms, unless otherwise indicated, shall be understood to have the following meanings:

“Clinically proven effective” mean that the results of clinical trial are statistically significant, i.e., the results of the clinical trial are not likely to be due to chance with an alpha level less than 0.05.

“ITT population” means all patients who were randomized and took at least one dose of study medication.

“Patient” means mammals, particularly humans.

“Pharmaceutically effective amount” means an amount of a compound/composition according to the present invention effective in producing the desired therapeutic effect.

“Stable dose of glatiramer acetate” means administering, for example, about 20 mg glatiramer acetate per day, particularly by subcutaneous injection of about 20 mg once a day.

“Treat” or “treating” means to alleviate symptoms, eliminate the causation of the symptoms either on a temporary or permanent basis, or to prevent or slow the appearance of symptoms of the named disorder or condition.

One particular embodiment of the invention is a method for treating relapsing-remitting form of multiple sclerosis, in a patient in need thereof, comprising administering to the patient about 7 mg or about 14 mg of teriflunomide once a day, and a stable dose of glatiramer acetate.

Another particular embodiment of the invention is a method for treating relapsing-remitting form of multiple sclerosis, in a patient in need thereof, comprising concurrently administering to the patient about 7 mg or about 14 mg of teriflunomide once a day, and a stable dose of glatiramer acetate.

Another particular embodiment of the invention is related to a clinically proven effective method for treating relapsing-remitting form of multiple sclerosis, in a patient in need thereof, comprising administering to the patient about 7 mg or about 14 mg of teriflunomide once a day, and a stable dose of glatiramer acetate.

Another particular embodiment of the invention is related to a clinically proven effective method for treating relapsing-remitting form of multiple sclerosis, in a patient in need thereof, comprising concurrently administering to the patient about 7 mg or about 14 mg of teriflunomide once a day, and a stable dose of glatiramer acetate.

Another particular embodiment of the invention is related to a method for treating relapsing-remitting form of multiple sclerosis, in a patient in need thereof, comprising administering to the patient about 7 mg of teriflunomide once a day and about 20 mg of glatiramer acetate daily.

Another particular embodiment of the invention is related to a method for treating relapsing-remitting form of multiple sclerosis, in a patient in need thereof, comprising administering to the patient about 14 mg of teriflunomide once a day and about 20 mg of glatiramer acetate daily.

Another particular embodiment of the invention is related to a method for treating relapsing-remitting form of multiple sclerosis, wherein the teriflunomide is administered orally.

Another particular embodiment of the invention is related to a method for treating relapsing-remitting form of multiple sclerosis, wherein the glatiramer acetate is administered subcutaneously.

Another particular embodiment of the invention is related to a method for reducing the number of T1-Gd lesions in a patient afflicted with relapsing-remitting form of multiple sclerosis, comprising administering to the patient about 7 mg or about 14 mg of teriflunomide, and a pharmaceutically effective amount of glatiramer acetate.

Another particular embodiment of the invention is related to a method for reducing the number of T1-Gd lesions in a patient afflicted with relapsing-remitting form of multiple sclerosis, comprising administering to the patient about 7 mg or about 14 mg of teriflunomide once a day, and a stable dose of glatiramer acetate.

Another particular embodiment of the invention is related to a clinically proven effective method for reducing the number of T1-Gd lesions in a patient afflicted with relapsing-remitting form of multiple sclerosis, comprising administering to the patient about 7 mg of teriflunomide once a day, and a stable dose of glatiramer acetate.

Another particular embodiment of the invention is related to a clinically proven effective method for reducing the number of T1-Gd lesions in a patient afflicted with relapsing-remitting form of multiple sclerosis, comprising concurrently administering to the patient about 7 mg of teriflunomide once a day, and a stable dose of glatiramer acetate.

Another particular embodiment of the invention is related to a method for reducing the number of T1-Gd lesions in a patient afflicted with multiple sclerosis, comprising administering to the patient about 7 mg or 14 mg of teriflunomide or a therapeutically equivalent amount of a pharmaceutically acceptable salt thereof, and a pharmaceutically effective amount of glatiramer acetate, wherein more number of T1-Gd lesions is reduced in the patient than in a patient treated by a stable dose of glatiramer acetate alone.

Another particular embodiment of the invention is related to a clinically proven effective method for reducing numbers of T1-Gd lesions in patients afflicted with relapsing-remitting form of multiple sclerosis, comprising administering to the patients about 7 mg of teriflunomide once a day, and a stable dose of glatiramer acetate, wherein more numbers of T1-Gd lesions are reduced in the patients treated by the method than in patients treated by a stable dose of glatiramer acetate alone.

Another particular embodiment of the invention is related to a method for reducing the number of T1-Gd lesions in patients afflicted with relapsing-remitting form of multiple sclerosis, comprising administering to the patient about 7 mg of teriflunomide once a day, and a stable dose of glatiramer acetate, wherein the number of T1-Gd lesions in the patients is reduced about 64% to about 70.2% comparing to the number of lesions in patients treated by a stable dose of glatiramer acetate.

Another particular embodiment of the invention is related to a method for reducing the number of T1-Gd lesions in patients afflicted with relapsing-remitting form of multiple sclerosis, comprising administering to the patient about 14 mg of teriflunomide once a day, and a stable dose of glatiramer acetate, wherein the number of T1-Gd lesions in the patients is reduced about 46.6% to about 53.6% comparing to the number of lesions in patients treated by a stable dose of glatiramer acetate.

Another particular embodiment of the invention is related to a method for reducing the volume of T1-Gd lesions in a patient afflicted with relapsing-remitting form of multiple sclerosis, comprising administering to the patient about 7 mg or about 14 mg of teriflunomide, and a pharmaceutically effective amount of glatiramer acetate.

Another particular embodiment of the invention is related to a method for reducing the volume of T1-Gd lesions in a patient afflicted with relapsing-remitting form of multiple sclerosis, comprising administering to the patient about 7 mg or about 14 mg of teriflunomide once a day, and a stable dose of glatiramer acetate.

Another particular embodiment of the invention is related to a method for reducing the volume of T1-Gd lesions in a patient afflicted with relapsing-remitting form of multiple sclerosis, comprising concurrently administering to the patient about 7 mg or about 14 mg of teriflunomide once a day, and a stable dose of glatiramer acetate.

Another particular embodiment of the invention is related to a clinically proven effective method for reducing the volume of T1-Gd lesions in patients afflicted with relapsing-remitting form of multiple sclerosis, comprising administering to the patients about 14 mg of teriflunomide once a day, and a stable dose of glatiramer acetate.

Another particular embodiment of the invention is related to a clinically proven effective method for reducing the volume of T1-Gd lesions in patients afflicted with relapsing-remitting form of multiple sclerosis, comprising concurrently administering to the patients about 14 mg of teriflunomide once a day, and a stable dose of glatiramer acetate.

Another particular embodiment of the invention is related to a method for reducing the volume of T1-Gd lesions in a patient afflicted with relapsing-remitting form of multiple sclerosis, comprising administering to the patient about 7 mg or about 14 mg of teriflunomide once a day, and a stable dose of glatiramer acetate, wherein more volume of T1-Gd lesions is reduced in the patient treated by the method than in a patient treated by a stable dose of glatiramer acetate.

Another particular embodiment of the invention is related to a clinically proven effective method for reducing the volume of T1-Gd lesions in patients afflicted with relapsing-remitting form of multiple sclerosis, comprising administering to the patients about 14 mg of teriflunomide once a day, and a stable dose of glatiramer acetate, wherein more volumes of T1-Gd lesions are reduced in the patients than in patients treated by a stable dose of glatiramer acetate.

Another particular embodiment of the invention is related to a method for reducing the volume of T1-Gd lesions in patients afflicted with relapsing-remitting form of multiple sclerosis, comprising administering to the patient about 7 mg teriflunomide once a day, and a stable dose of glatiramer acetate, wherein the volume of T1-Gd lesions in the patients treated by the method is reduced about 40.4% to about 55.6% comparing to the volume of lesions in patients treated by a stable dose of glatiramer acetate.

Another particular embodiment of the invention is related to a method for reducing the volume of T1-Gd lesions in patients afflicted with relapsing-remitting form of multiple sclerosis, comprising administering to the patient about 14 mg of teriflunomide once a day, and a stable dose of glatiramer acetate, wherein the volume of T1-Gd lesions in the patients treated by the method is reduced about 73.0% to about 73.1% comparing to the volume of lesions in patients treated by a stable dose of glatiramer acetate.

Another particular embodiment of the invention is related to a method for treating relapsing-remitting form of multiple sclerosis in patients in need thereof comprising administering to the patients 14 mg of teriflunomide once a day and a stable dose of glatiramer acetate, wherein about 81.6% of the patients are free of T1-Gd lesions after about 24-week treatment.

Another particular embodiment of the invention is related to a method for treating relapsing-remitting form of multiple sclerosis in patients in need thereof comprising administering to the patients 14 mg of teriflunomide once a day and a stable dose of glatiramer acetate, wherein about 78.9% of the patients are free of T1-Gd lesions after about 48-week treatment.

Another particular embodiment of the invention related to a method for reducing annualized relapse rate in a patient afflicted with relapsing-remitting form of multiple sclerosis, comprising administering to the patient about 7 mg or about 14 mg of teriflunomide once a day, and a stable dose of glatiramer acetate.

Another particular embodiment of the invention related to a method for reducing annualized relapse rate in a patient afflicted with relapsing-remitting form of multiple sclerosis, comprising concurrently administering to the patient about 7 mg or about 14 mg of teriflunomide once a day, and a stable dose of glatiramer acetate.

Another particular embodiment of the invention related to a method for reducing annualized relapse rate in a patient afflicted with relapsing-remitting form of multiple sclerosis, comprising concurrently administering to the patient about 7 mg of teriflunomide once a day, and a stable dose of glatiramer acetate, wherein the patient treated by the method has an annualized relapse rate lower than a patient treated by a stable dose of glatiramer acetate alone.

Another particular embodiment of the invention related to a method for reducing annualized relapse rate in a patient afflicted with relapsing-remitting form of multiple sclerosis, comprising concurrently administering to the patient about 7 mg of teriflunomide once a day, and a stable dose of glatiramer acetate, wherein the annualized relapse rate of the patient treated by the method is reduced by about 34.5% to about 37.8% comparing to the annualized relapse rate of a patient treated by a stable dose of glatiramer acetate alone.

Another particular embodiment of the invention is related to the use of about 7 mg or about 14 mg of teriflunomide for the preparation of a medicament for treating relapsing-remitting form of multiple sclerosis, wherein said medicament is administered to a patient in combination of a pharmaceutically effective amount of glatiramer acetate.

Another particular embodiment of the invention is related to the use of about 7 mg or about 14 mg of teriflunomide for the preparation of a medicament for treating relapsing-remitting form of multiple sclerosis, wherein said medicament is administered to a patient once a day in combination of a stable of glatiramer acetate.

Another particular embodiment of the invention is related to the use of about 7 mg or about 14 mg of teriflunomide for the preparation of a medicament for treating relapsing-remitting form of multiple sclerosis, wherein said medicament is administered once a day to a patient who is concurrently on a stable of glatiramer acetate.

Another particular embodiment of the invention is related to the use of about 7 mg or about 14 mg teriflunomide for the preparation of a clinically proven effective medicament for treating relapsing-remitting form of multiple sclerosis, wherein said medicament is administered once a day in combination of a stable dose of glatiramer acetate.

Another particular embodiment of the invention is related to the use of about 7 mg or about 14 mg of teriflunomide for the preparation of a clinically proven effective medicament for treating relapsing-remitting form of multiple sclerosis, wherein said medicament is administered once a day to a patient who is concurrently on a stable dose of glatiramer acetate.

Another particular embodiment of the invention is related to the use of about 7 mg or about 14 mg of teriflunomide for the preparation of a medicament for reducing the number of T1-Gd lesions in a patient afflicted with relapsing-remitting form of multiple sclerosis, wherein said medicament is administered once a day to the patient in combination of a pharmaceutically effective amount of glatiramer acetate.

Another particular embodiment of the invention is related to the use of about 7 mg or about 14 mg of teriflunomide for the preparation of a medicament for reducing the number of T1-Gd lesions in a patient afflicted with relapsing-remitting form of multiple sclerosis, wherein said medicament is administered once a day to the patient in combination of a stable dose of glatiramer acetate.

Another particular embodiment of the invention is related to the use of about 7 mg of teriflunomide for the preparation of a clinically proven effective medicament for reducing the number of T1-Gd lesions in a patient afflicted with relapsing-remitting form of multiple sclerosis, wherein said medicament is administered once a day to the patient in combination of a stable dose of glatiramer acetate.

Another particular embodiment of the invention is related to the use of about 7 mg of teriflunomide for the preparation of a clinically proven effective medicament for reducing the number of T1-Gd lesions in a patient afflicted with relapsing-remitting form of multiple sclerosis, wherein said medicament is administered once a day to the patient who is concurrently on a stable dose of glatiramer acetate.

Another particular embodiment of the invention is related to the use of about 7 mg or about 14 mg of teriflunomide for the preparation of a medicament for reducing the number of T1-Gd lesions in a patient afflicted with relapsing-remitting form of multiple sclerosis, wherein said medicament is administered once a day to the patient in combination of a stable dose of glatiramer acetate such that more number of T1-Gd lesions are reduced in the patient than in a patient treated by a stable dose of glatiramer acetate alone.

Another particular embodiment of the invention is related to the use of about 7 mg of teriflunomide for the preparation of a clinically proven effective medicament for reducing the number of T1-Gd lesions in a patient afflicted with relapsing-remitting form of multiple sclerosis, wherein said medicament is administered once a day to the patient in combination of a stable dose of glatiramer acetate such that more number of T1-Gd lesions are reduced in the patient than in a patient treated by a stable dose of glatiramer acetate alone.

Another particular embodiment of the invention is related to the use of about 7 mg teriflunomide for the preparation of a medicament for reducing the number of T1-Gd lesions in patients afflicted with relapsing-remitting form of multiple sclerosis, wherein said medicament is administered once a day to the patients in combination of a stable dose of glatiramer acetate such that the number of T1-Gd lesions in the patients is reduced about 64% to about 70.2% comparing to the number of lesions in patients treated by a stable dose of glatiramer acetate alone.

Another particular embodiment of the invention is related to the use of about 14 mg teriflunomide for the preparation of a medicament for reducing the number of T1-Gd lesions in patients afflicted with relapsing-remitting form of multiple sclerosis, wherein said medicament is administered once a day to the patients in combination of a stable dose of glatiramer acetate such that the number of T1-Gd lesions in the patients is reduced about 46.6% to about 53.6% comparing to the number of lesions in patients treated by a stable dose of glatiramer acetate alone.

Another particular embodiment of the invention is related to the use of about 7 mg or about 14 mg of teriflunomide for the preparation of a medicament for reducing the volume of T1-Gd lesions in a patient afflicted with relapsing-remitting form of multiple sclerosis, wherein said medicament is administered once a day to the patient in combination of a pharmaceutically effective amount of glatiramer acetate.

Another particular embodiment of the invention is related to the use of about 7 mg or about 14 mg of teriflunomide for the preparation of a medicament for reducing the volume of T1-Gd lesions in a patient afflicted with relapsing-remitting form of multiple sclerosis, wherein said medicament is administered once a day to the patient in combination of a stable dose of glatiramer acetate.

Another particular embodiment of the invention is related to the use of about 7 mg or about 14 mg of teriflunomide for the preparation of a medicament for reducing the volume of T1-Gd lesions in a patient afflicted with relapsing-remitting form of multiple sclerosis, wherein said medicament is administered once a day to the patient who is concurrently on a stable dose of glatiramer acetate.

Another particular embodiment of the invention is related to the use of about 14 mg of teriflunomide for the preparation of a clinically proven effective medicament for reducing the volume of T1-Gd lesions in a patient afflicted with relapsing-remitting form of multiple sclerosis, wherein said medicament is administered once a day to the patient in combination with a stable dose of glatiramer acetate.

Another particular embodiment of the invention is related to the use of about 14 mg teriflunomide for the preparation of a medicament for reducing the volume of T1-Gd lesions in a patient afflicted with relapsing-remitting form of multiple sclerosis, wherein said medicament is administered once a day to the patient who is concurrently on a stable dose of glatiramer acetate.

Another particular embodiment of the invention is related to the use of about 7 mg or about 14 mg of teriflunomide for the preparation of a medicament for reducing the volume of T1-Gd lesions in a patient afflicted with relapsing-remitting form of multiple sclerosis, wherein said medicament is administered once a day to the patient in combination of a stable dose of glatiramer acetate such that more volume of T1-Gd lesions are reduced in the patient than in a patient treated by a stable dose of glatiramer acetate.

Another particular embodiment of the invention is related to the use of about 14 mg of teriflunomide for the preparation of a clinically proven effective medicament for reducing the volume of T1-Gd lesions in patients afflicted with relapsing-remitting form of multiple sclerosis, wherein said medicament is administered once a day to the patients in combination of a stable dose of glatiramer acetate such that more volume of T1-Gd lesions are reduced in the patients than in patients treated by a stable dose of glatiramer acetate.

Another particular embodiment of the invention is related to the use of about 7 mg teriflunomide for the preparation of a medicament for reducing the volume of T1-Gd lesions in patients afflicted with relapsing-remitting form of multiple sclerosis, wherein said medicament is administered once a day to the patients in combination of a stable dose of glatiramer acetate such that the volume of T1-Gd lesions in the patients is reduced about 40.4% to about 55.6% comparing to the number of lesions in patients treated by a stable dose of glatiramer acetate alone.

Another particular embodiment of the invention is related to the use of about 14 mg teriflunomide for the preparation of a medicament for reducing the volume of T1-Gd lesions in patients afflicted with relapsing-remitting form of multiple sclerosis, wherein said medicament is administered once a day to the patients in combination of a stable dose of glatiramer acetate such that the volume of T1-Gd lesions in the patients is reduced about 73.0% to about 73.1% comparing to the number of lesions in patients treated by a stable dose of glatiramer acetate alone.

Another particular embodiment of the invention is related to the use of about 14 mg of teriflunomide for the preparation of a medicament for treating relapsing-remitting form of multiple sclerosis, wherein said medicament is administered once a day to patients in combination of a stable dose of glatiramer acetate such that about 81.6% of the patients are free of T1-Gd lesions after about 24-week treatment.

Another particular embodiment of the invention is related to the use of about 14 mg teriflunomide of for the preparation of a medicament for treating relapsing-remitting form of multiple sclerosis, wherein said medicament is administered once a day to patients in combination of a stable dose of glatiramer acetate such that about 78.9% of the patients are free of T1-Gd lesions after about 48-week treatment.

Another particular embodiment of the invention is related to the use of about 7 mg or about 14 mg of teriflunomide for the preparation of a medicament for reducing annualized relapse rate in a patient afflicted with relapsing-remitting form of multiple sclerosis, wherein said medicament is administered once a day to the patient in combination of a stable dose of glatiramer acetate.

Another particular embodiment of the invention is related to the use of about 7 mg or about 14 mg of teriflunomide for the preparation of a medicament for reducing annualized relapse rate in a patient afflicted with relapsing-remitting form of multiple sclerosis, wherein said medicament is administered once a day to the patient who is concurrently on a stable dose of glatiramer acetate.

Another particular embodiment of the invention is related to the use of about 7 mg of teriflunomide for the preparation of a medicament for reducing annualized relapse rate in a patient afflicted with relapsing-remitting form of multiple sclerosis, wherein said medicament is administered once a day to the patient who is concurrently on a stable dose of glatiramer acetate such that the patient has an annualized relapse rate lower than a patient treated by a stable dose of glatiramer acetate alone.

Another particular embodiment of the invention is related to the use of about 7 mg of teriflunomide for the preparation of a medicament for reducing annualized relapse rate in a patient afflicted with relapsing-remitting form of multiple sclerosis, wherein said medicament is administered once a day to the patient who is concurrently on a stable dose of glatiramer acetate such that the annualized relapse rate of the patient is reduced by about 34.5% to about 37.8% comparing to the annualized relapse rate of a patient treated by a stable dose of glatiramer acetate alone.

It is now found that the addition of teriflunomide to stable-dosed glatiramer acetate improves disease control beyond glatiramer acetate alone, in terms of MRI activity, without added concerns regarding safety and tolerability.

### EXAMPLES

The present invention may be better understood by reference to the following non-limiting Examples, which are exemplary of the invention. They should in no way be construed, however, as limiting the broad scope of the invention.

### Example 1

A placebo-controlled, double-blinded, randomized study was conducted in relapsing-remitting multiple sclerosis patients who were concurrently on a stable dose of glatiramer acetate. The dose of glatiramer acetate was 20 mg subcutaneously daily.

Around 40 patients were treated in each treatment group (placebo: 41, 7 mg: 42, and 14 mg: 40). The demographic and disease baseline characteristics were generally comparable amount the 3 treatment groups. The mean age of the study population was 41.4 years. The majority of patients were female (78.9%) and had a relapsing remitting type of MS 94.3%), with a diagnosis of MS since around 8 years prior and approximately 41% of patients had no relapse in the previous year. The base line Expanded Disability Status Scale (EDSS) score was similar between treatment groups (around 2.5). A few more patients had Magnetic Resonance Image (MRI) disease activity (at least one T1-Gd lesion) at baseline in 7 mg group (28.6%) than in placebo (14.6%) and 14 mg (12.8%) groups.

Method:

Safety was evaluated with reported Treatment Emergent Adverse Events (TEAE), physical examination (every 8-weeks), laboratory data (e.g. Liver Function Test, every 2 weeks), vital signs (every 8-weeks), ECG and abdominal ultrasound (at baseline and at 24-weeks). Brain MRI activity, including T1-gadolinium (T1-Gd) lesions with central reading was recorded every 8-weeks, EDSS scores were recorded at baseline and at 24-weeks, Fatigue impact scale was assessed every 8-weeks, and relapses were assessed at every clinical visit.

Results:

Safety: Tolerability and safety of each teriflunomide dose combined with glatiramer acetate were acceptable during the 24-week study (TEAE incidence rates: placebo: 78.0%, 7 mg: 83.3%, 14 mg: 87.5%). The number of patients with TEAE leading to treatment discontinuation was 0 in placebo; 3 (7.1%) in 7 mg; and 4 (10%) in 14 mg. Around 90% of patients completed the 24-week treatment period (placebo: 95.1%, 7 mg: 88.1%, and 14 mg: 85.0%).

The proportion of patients with ALT (alanine aminotransferase) greater than 3ULN (three times above the Upper Limit of Normal range for the central laboratory) was low and similar across treatment groups (placebo: 1; 7 mg: 0; 14 mg: 1). The proportion of patients with a TEAE related to hepatic disorder was slightly higher in the 14 mg group as compared to the other groups (placebo: 12.5%, 7 mg: 9.5%, and 14 mg: 12.2%), and no dose effect was shown No case of concurrent increase in ALT>3ULN and in total bilirubin>2ULN was observed.

The proportion of patients with TEAE potentially related to immunosuppression was balanced for placebo (44%) and 7 mg group (43%) with slightly lower frequency in 14 mg group (38%). The proportion of patients with occurrence of decreased WBC (white blood cell) count and neutrophils (PCSA) was slightly higher in the two teriflunomide groups.

No patients died in this study.

Efficacy: The proportion of patient with at least one T1-Gd lesion on the baseline MRI scan was unbalanced among the groups, with a greater number of patients with T1-Gd activity in 7 mg group than in placebo and 14 mg groups (14.6% in placebo, 28.6% in 6 mg, and 12.8% in 14 mg), as was the number of lesion per patient (baseline-scan) (0.220 in placebo, 0.738 in 7 mg, and 0.333 in 14 mg).

As shown in Table 1, over 24 weeks, the unadjusted number of lesions per scan decreased in both teriflunomide groups and increased in placebo (0.461 in placebo, 0.321 in 7 mg and 0.262 in 14 mg). This was seen also in the mean number of lesions per patient (“Patient Gd-enhancing T1-lesions per scan”).

Similarly the main analysis on number of T1-Gd lesions over 24 weeks using Poisson model with regions and baseline number of lesions as covariates indicated a relative risk reduction of 70.2% at 7 mg (p=0.0110) and 53.6% at 14 mg (p=0.1157).

This drug effect was reflected by the greater number of patients without Gd-enhancing lesions over 24-week at 14 mg (73.2% in placebo and 81.6% in 14 mg). The higher baseline activity in 7 mg group may have contributed to the observed smaller number of patients without activity (61.0%).

Analysis of total volume (ml) of Gd-enhancing T1-lesions per MRI scan using a permutation test indicated that the volume of T1-Gd lesions was reduced at 7 mg (p=0.0886, relative reduction versus placebo: 55.6%) and 14 mg (p=0.0395, relative reduction versus placebo: 73.0%) (Table 2).

Twenty-nine patients experienced a total of 30 confirmed relapses. Two relapses occurred after treatment discontinuation (1 day in one placebo patient, 41 days in one 7 mg patient). Therefore, 28 confirmed relapses occurring in 27 patients were included in the analysis (Table 3).

Ten relapses were observed in the placebo, 6 in the 7 mg group and 12 in the 14 mg group, corresponding to an unadjusted annualized relapse rate (ARR) of 0.535 per patient year in placebo, 0.337 in teriflunomide 7 mg, and 0.710 in teriflunomide 14 mg. The adjusted ARR from the Poisson regression model was 0.475 in placebo, 0.311 in teriflunomide 7 mg, and 0.647 in teriflunomide 14 mg. This represented a 34.5% relative decrease in the ARR at 7 mg and 36.2% increase at 14 mg versus placebo.

Conclusion: The addition of teriflunomide to stable-dosed glatiramer acetate improved disease control beyond glatiramer acetate alone, as evaluated by T1-Gd MRI activity, with acceptable safety and tolerability over 24 weeks of treatment.

TABLE 1
Analysis of total number of Gd-enhancing T1-lesions per MRI scan - ITT population

| | | | teriflunomide | | | |
|---|---|---|---|---|---|---|
| | | | 7 mg | | 14 mg | |
| | Placebo + GA | | teri + GA | | teri + GA | |
| | (N = 41) | | (N = 42) | | (N = 40) | |
| Baseline | | | | | | |
| Patients with >=1 Gd- | | | | | | |
| enhancing T1-lesions | | | | | | |
| Yes | 6 | (14.6%) | 12 | (28.6%) | 5 | (12.8%) |
| No | 35 | (85.4%) | 30 | (71.4%) | 34 | (87.2%) |
| Patient Gd-enhancing T1- | | | | | | |
| lesions | | | | | | |
| N | 41 | | 42 | | 39 | |
| Mean (SD) | 0.220 | (0.613) | 0.738 | (1.563) | 0.333 | (1.060) |
| Median | 0.000 | | 0.000 | | 0.000 | |
| Min:Max | 0.00:3.00 | | 0.00:7.00 | | 0.00:5.00 | |
| Post-baseline | | | | | | |
| Patients with >=1 Gd- | | | | | | |
| enhancing T1-lesions | | | | | | |
| Yes | 11 | (26.8%) | 16 | (39.0%) | 7 | (18.4%) |
| No | 30 | (73.2%) | 25 | (61.0%) | 31 | (81.6%) |
| P-value^{a} | | | 0.3474 | | 0.4293 | |
| Number of Gd-enhancing T1- | | | | | | |
| lesions | | | | | | |
| 0 | 30 | (73.2%) | 25 | (61.0%) | 31 | (81.6%) |
| 1 | 5 | (12.2%) | 11 | (26.8%) | 3 | (7.9%) |
| 2 | 1 | (2.4%) | 1 | (2.4%) | 2 | (5.3%) |
| 3 | 0 | | 2 | (4.9%) | 0 | |
| >=4 | 5 | (12.2%) | 2 | (4.9%) | 2 | (5.3%) |
| Total number of Gd-enhancing | 53 | | 36 | | 28 | |
| T1-lesions | | | | | | |
| Total number of scans | 115 | | 112 | | 107 | |
| Unadjusted Gd-enhancing T1- | 0.461 | | 0.321 | | 0.262 | |
| lesions per scan^{b} | | | | | | |
| Adjusted Gd-enhancing T1- | | | | | | |
| lesions per scan^{c} | | | | | | |
| Estimate (95% CI) | 0.367 | (0.183, 0.736) | 0.109 | (0.054, 0.220) | 0.171 | (0.093, 0.313) |
| Relative risk (95% CI) | | | 0.298 | (0.117, 0.757) | 0.464 | (0.179, 1.208) |
| P-value | | | 0.0110 | | 0.1157 | |
| Patient Gd-enhancing T1- | | | | | | |
| lesions per scan^{d} | | | | | | |
| N | 41 | | 41 | | 38 | |
| Mean (SD) | 0.459 | (1.197) | 0.329 | (0.759) | 0.250 | (0.870) |

| | | | |
|---|---|---|---|
| Median | 0.000 | 0.000 | 0.000 |
| Min:Max | 0.00:5.00 | 0.00:4.33 | 0.00:5.00 |

Note:
MRI: Magnetic resonance imaging.
^{a}From Fisher's exact test.
^{b}The total number of Gd-enhancing T1-lesion that occurred during the study divided by the total number of scans during the study.
^{c}Poisson model with the total number of Gd-enhancing T1-lesions as the response variable, baseline number of Gd-enhancing T1-lesions, treatment, and region as covariates, and log-transformed number of scans as an offset variable.
^{d}The number of of Gd-enhancing T1-lesions for each patient divided by the number of scans for that patient.

TABLE 2
Analysis of total volume (ml) of Gd-enhancing T1-lesions per MRI scans -
ITT population - ITT population

| | | teriflunomide | |
|---|---|---|---|
| | | 7 mg | 14 mg |
| | Placebo + GA | teri + GA | teri + GA |
| | (N = 41) | (N = 42) | (N = 40) |
| Baseline | | | |
| N | 41 | 42 | 39 |
| Mean (SD) | 0.011 (0.036) | 0.065 (0.196) | 0.057 (0.192) |
| Median | 0.000 | 0.000 | 0.000 |
| Min:Max | 0.00:0.18 | 0.00:0.88 | 0.00:1.01 |
| Post-baseline | | | |
| Gd-enhancing T1- | 0.063 | 0.028 | 0.017 |
| lesions per scan^{a} | | | |
| Patient Gd-enhancing | | | |
| T1-lesions per scan^{b} | | | |
| N | 41 | 41 | 38 |
| Mean (SD) | 0.064 (0.187) | 0.028 (0.057) | 0.017 (0.061) |
| Median | 0.000 | 0.000 | 0.000 |
| Min:Max | 0.00:0.89 | 0.00:0.26 | 0.00:0.35 |
| P-value^{c} | | 0.0886 | 0.0395 |

Note:
MRI: Magnetic resonance imaging
^{a}The total volume of Gd-enhancing T1-lesion that occurred during the study divided by the total number of scans during the study.
^{b}The volume of Gd-enhancing T1-lesions for each patient divided by the number of scans for that patient.
^{c}Permutation test using ANCOVA analysis adjusting for baseline volume of Gd-enhancing T1-lesions, treatment, and region.

TABLE 3
Analysis of MS relapse - ITT population

| | | | teriflunomide | | | |
|---|---|---|---|---|---|---|
| | | | 7 mg | | 14 mg | |
| | Placebo + GA | | teri + GA | | teri + GA | |
| | (N = 41) | | (N = 42) | | (N = 40) | |
| Number of patients with | | | | | | |
| >=1 relapses | | | | | | |
| Yes | 9 | (22.0%) | 6 | (14.3%) | 12 | (30.0%) |
| No | 32 | (78.0%) | 36 | (85.7%) | 28 | (70.0%) |
| Number of relapses | | | | | | |
| 0 | 32 | (78.0%) | 36 | (85.7%) | 28 | (70.0%) |
| 1 | 8 | (19.5%) | 6 | (14.3%) | 12 | (30.0%) |
| 2 | 1 | (2.4%) | 0 | | 0 | |
| 3 | 0 | | 0 | | 0 | |
| >=4 | 0 | | 0 | | 0 | |
| Total number of relapses | 10 | | 6 | | 12 | |
| Total patient-years | 18.7 | | 17.8 | | 16.9 | |
| followed | | | | | | |
| Unadjusted annualized | 0.535 | | 0.337 | | 0.710 | |
| relapses rate^{a} | | | | | | |
| Adjusted annualized | | | | | | |
| relapse rate^{b} | | | | | | |
| Estimate (95% CI) | 0.475 | (0.260, 0.867) | 0.311 | (0.151, 0.642) | 0.647 | (0.379, 1.103) |
| Relative risk (95% CI) | | | 0.655 | (0.258, 1.666) | 1.362 | (0.641, 2.897) |
| P-value | | | 0.3742 | | 0.4215 | |
| Patient annualized | | | | | | |
| replase rate^{c} | | | | | | |
| N | 41 | | 42 | | 40 | |
| Mean (SD) | 0.545 | (1.091) | 0.569 | (2.096) | 0.721 | (1.166) |

| | | | |
|---|---|---|---|
| Median | 0.000 | 0.000 | 0.000 |
| Min:Max | 0.00:4.35 | 0.00:13.04 | 0.00:4.35 |

^{a}The total number of confirmed relapses that occurred during the study divided by the total number of patient-years followed in the study.
^{b}Poisson model with the total number of confirmed relapses onset between randomization date and last dose date as the response variable, treatment and region as covariates, and log-transformed standardized study duration as an offset variable.
^{c}The number of confirmed relapses for each patient divided by the number of years followed in the study for that patient.

### Example 2

Design/Methods: 96 of the 123 patients that were randomized to study treatment (placebo: 40; 7 mg: 42; 14 mg: 41) for the first 6-months in Example 1 completed this 6-month period and accepted to continue medication for an additional 6-month period (placebo: 37, 7 mg: 30, 14 mg: 29). Of the 96 treated patients, 5 patients prematurely withdrew from the study treatment (placebo: 3, 7 mg: 0, and 14 mg: 2). Safety was evaluated from TEAE, physical exam (every 6 weeks), laboratory data (every 6 weeks), vital signs (every 6 weeks), ECG (at the close-out visit), pancreatic ultrasound (at the close-out visit) and brain MRI (at the close-out visit). Relapses were recorded and EDSS was performed every 6-weeks. Annualized relapse rates (ARR) were analyzed by a Poisson model with the total number of confirmed relapses onset between randomization date and last dose date as the response variable, treatment and region as covariates, and log-transformed standardized study duration as an offset variable. The following results report the entire 48-week double-blind treatment period.

Result:

Safety: Tolerability and safety of each dose of teriflunomide combined with glatiramer acetate were acceptable and manageable during the 48-week study (TEAE incidence rates: placebo: 97.5%, 7 mg: 95.2%, 14 mg: 92.7%). The number of patients with TEAE leading to treatment discontinuation was 2 (5.0%) in placebo, 3 (7.1%) in 7 mg and 5 (12.2%) in 14 mg. The number of patients experiencing a serious TEAE was 6 (15.0%) in placebo, 5 (11.9%) in 7 mg, and 2 (4.9%).

The proportion of patients with ALT greater than 3×ULN was low and similar across treatment groups (placebo: 1; 7 mg: 0; 14 mg: 1). The proportion of patients with a TEAE related to hepatic disorder was generally balanced between groups (placebo: 12.5%, 7 mg: 9.5%, and 14 mg: 12.2%), and no dose effect was shown.

The proportion of patients with a TEAE potentially related to immunosuppression (grouping all infections and hematological disorders) was higher in the placebo group (67.5%) than the two teriflunomide groups (52.4% in 7 mg and 51.2% in 14 mg). The proportions of patients with occurrence of decreased WBC and neutrophil counts (PCSA) were slightly higher in the two teriflunomide groups.

The proportion of patients experiencing a TEAE related to hypersensitivity was higher in the two teriflunomide groups (14.3% in 7 mg, and 24.4% in 14 mg) compared to the placebo group (10.0%) with an apparent dose effect.

Four patients experienced a TEAE related to hypertension; 0 in placebo, 2 in 7 mg and 2 in 14 mg.

Efficacy: A summary of total number of (gadolinium) Gd-enhancing T1-lesions for the ITT population is presented in Table 4. One patient (14 mg group) had one day participation in the study and had no valid baseline MRI data. Two other patients, one in 7 mg and one in 14 mg, prematurely discontinued the study and no post baseline MRI was collected.

The proportion of patients with at least one Gd-T1 lesion on the baseline MRI scan was unbalanced among the groups, with a greater number of patients with T1-Gd activity in 7 mg group than in placebo and 14 mg groups (14.6% in placebo, 28.6% in 7 mg and 12.8% in 14 mg), as was the mean number of lesion per patient (baseline-scan) (0.220 in placebo, 0.738 in 7 mg and 0.333 in 14 mg).

Over 48 weeks, the unadjusted number of lesions per scan decreased in both teriflunomide groups and increased in placebo (0.392 in placebo, 0.317 in 7 mg and 0.233 in 14 mg). This was seen also in the mean number of lesions per patient (“Patient Gd-enhancing T1-lesions per scan”).

Similarly the main analysis on number of T1-Gd lesions over 48 weeks using Poisson model with regions and baseline number of lesions as covariates indicated a relative risk reduction of 64% at 7 mg (p=0.0306) and 46.6% at 14 mg (p=0.1931).

This drug effect was also reflected by the greater number of patients without Gd-enhancing lesions over 48-week at 14 mg (68.3% in placebo and 78.9% in 14 mg). The higher baseline activity in 7 mg group may have contributed to the observed smaller number of patients without activity (56.1%).

Analysis of total volume (ml) of Gd-enhancing T1-lesions per MRI scan using a permutation test indicated that the volume of T1-Gd lesions was reduced at 7 mg (p=0.1340, relative reduction versus placebo: 40.4%) and 14 mg (p=0.0381, relative reduction versus placebo: 73.1%) (Table 5).

During the study treatment period, there were 38 patients with at least one relapse (15 on placebo, 8 on 7 mg, and 15 on 14 mg, see Table 6), corresponding to an unadjusted annualized relapse rate (ARR) of 0.473 per patient year in placebo, 0.281 in teriflunomide 7 mg, and 0.546 in teriflunomide 14 mg. The adjusted ARR from the Poisson regression model is 0.420 in placebo, 0.262 in teriflunomide 7 mg, and 0.497 in teriflunomide 14 mg. This represents a 37.8% relative decrease in the ARR at 7 mg and an 18.3% relative increase at 14 mg versus placebo.

Conclusion: In this one-year study, the safety of the addition of teriflunomide to glatiramer acetate appears manageable; there were no additional concerns since the 24 weeks of treatment. There was a trend to benefit on controlling disease activity in this phase II safety study extension as measured by T1-Gd lesions.

TABLE 4
Total number of Gd-enhancing T1-lesions per MRI scan - ITT population

| | | | teriflunomide | | | |
|---|---|---|---|---|---|---|
| | | | 7 mg | | 14 mg | |
| | Placebo + GA | | teri + GA | | teri + GA | |
| | (N = 41) | | (N = 42) | | (N = 40) | |
| Baseline | | | | | | |
| Patients with >=1 Gd- | | | | | | |
| enhancing T1-lesions | | | | | | |
| Yes | 6 | (14.6%) | 12 | (28.6%) | 5 | (12.8%) |
| No | 35 | (85.4%) | 30 | (71.4%) | 34 | (87.2%) |
| Patient Gd-enhancing T1- | | | | | | |
| lesions | | | | | | |
| N | 41 | | 42 | | 39 | |
| Mean (SD) | 0.220 | (0.613) | 0.738 | (1.563) | 0.333 | (1.060) |
| Median | 0.000 | | 0.000 | | 0.000 | |
| Min:Max | 0.00:3.00 | | 0.00:7.00 | | 0.00:5.00 | |
| Post-baseline | | | | | | |
| Patients with >=1 Gd- | | | | | | |
| enhancing T1-lesions | | | | | | |
| Yes | 13 | (31.7%) | 18 | (43.9%) | 8 | (21.1%) |
| No | 28 | (68.3%) | 23 | (56.1%) | 30 | (78.9%) |
| P-value^{a} | | | 0.3625 | | 0.3182 | |
| Number of Gd-enhancing | | | | | | |
| T1-lesions | | | | | | |
| 0 | 28 | (68.3%) | 23 | (56.1%) | 30 | (78.9%) |
| 1 | 6 | (14.6%) | 12 | (29.3%) | 3 | (7.9%) |
| 2 | 2 | (4.9%) | 2 | (4.9%) | 2 | (5.3%) |
| 3 | 0 | | 0 | | 1 | (2.6%) |
| >=4 | 5 | (12.2%) | 4 | (9.8%) | 2 | (5.3%) |
| Total number of Gd- | 60 | | 45 | | 31 | |
| enhancing T1-lesions | | | | | | |
| Total number of scans | 153 | | 142 | | 133 | |
| Unadjusted Gd-enhancing | 0.392 | | 0.317 | | 0.233 | |
| T1-lesions per scan^{b} | | | | | | |
| Adjusted Gd-enhancing T1- | | | | | | |
| lesions per scan^{c} | | | | | | |
| Estimate (95% CI) | 0.333 | (0.171, 0.649) | 0.120 | (0.059, 0.243) | 0.178 | (0.098, 0.324) |
| Relative risk (95% CI) | | | 0.360 | (0.143, 0.909) | 0.534 | (0.208, 1.373) |
| P-value | | | 0.0306 | | 0.1931 | |
| Patient Gd-enhancing T1- | | | | | | |
| lesions per scan^{d} | | | | | | |
| N | 41 | | 41 | | 38 | |
| Mean (SD) | 0.409 | (1.017) | 0.313 | (0.655) | 0.257 | (0.870) |

| | | | |
|---|---|---|---|
| Median | 0.000 | 0.000 | 0.000 |
| Min:Max | 0.00:4.25 | 0.00:3.50 | 0.00:5.00 |

Note:
MRI: Magnetic resonance imaging.
^{a}From Fisher's exact test.
^{b}The total number of Gd-enhancing T1-lesion that occurred during the study divided by the total number of scans during the study.
^{c}Poisson model with the total number of Gd-enhancing T1-lesions as the response variable, baseline number of Gd-enhancing T1-lesions, treatment, and region as covariates, and log-transformed number of scans as an offset variable.
^{d}The number of of Gd-enhancing T1-lesions for each patient divided by the number of scans for that patient.

TABLE 5
Total volume (ml) of Gd-enhancing T1-lesions per MRI scan - ITT
population

| | | teriflunomide | |
|---|---|---|---|
| | | 7 mg | 14 mg |
| | Placebo + GA | teri + GA | teri + GA |
| | (N = 41) | (N = 42) | (N = 40) |
| Baseline | | | |
| N | 41 | 42 | 39 |
| Mean (SD) | 0.011 (0.036) | 0.065 (0.196) | 0.057 (0.192) |
| Median | 0.000 | 0.000 | 0.000 |
| Min:Max | 0.00:0.18 | 0.00:0.88 | 0.00:1.01 |
| Post-baseline | | | |
| Gd-enhancing | 0.052 | 0.031 | 0.014 |
| T1-lesions per scan^{a} | | | |
| Patient Gd-enhancing | | | |
| T1-lesions per scan^{b} | | | |
| N | 41 | 41 | 38 |
| Mean (SD) | 0.056 (0.162) | 0.030 (0.057) | 0.016 (0.059) |
| Median | 0.000 | 0.000 | 0.000 |
| Min:Max | 0.00:0.76 | 0.00:0.23 | 0.00:0.35 |
| P-value^{c} | | 0.1340 | 0.0381 |

Note:
MRI: Magnetic resonance imaging
^{a}The total volume of Gd-enhancing T1-lesion that occurred during the study divided by the total number of scans during the study.
^{b}The volume of of Gd-enhancing T1-lesions for each patient divided by the number of scans for that patient.
^{c}Permutation test using ANCOVA analysis adjusting for baseline volume of Gd-enhancing T1-lesions, treatment, and region.

TABLE 6
Analysis of MS relapses - ITT population

| | | | teriflunomide | | | |
|---|---|---|---|---|---|---|
| | | | 7 mg | | 14 mg | |
| | Placebo + GA | | teri + GA | | teri + GA | |
| | (N = 41) | | (N = 42) | | (N = 40) | |
| Number of patients with >=1 | | | | | | |
| relapses | | | | | | |
| Yes | 15 | (36.6%) | 8 | (19.0%) | 15 | (37.5%) |
| No | 26 | (63.4%) | 34 | (81.0%) | 25 | (62.5%) |
| Number of relapses | | | | | | |
| 0 | 26 | (63.4%) | 34 | (81.0%) | 25 | (62.5%) |
| 1 | 13 | (31.7%) | 7 | (16.7%) | 14 | (35.0%) |
| 2 | 2 | (4.9%) | 1 | (2.4%) | 1 | (2.5%) |
| 3 | 0 | | 0 | | 0 | |
| >=4 | 0 | | 0 | | 0 | |
| Total number of relapses | 17 | | 9 | | 16 | |
| Total patient-years followed | 36 | | 32 | | 29.3 | |
| Unadjusted annualized | 0.473 | | 0.281 | | 0.546 | |
| relapses rate^{a} | | | | | | |
| Adjusted annualized relapse | | | | | | |
| rate^{b} | | | | | | |
| Estimate (95% CI) | 0.420 | (0.270, 0.654) | 0.262 | (0.140, 0.489) | 0.497 | (0.316, 0.783) |
| Relative risk (95% CI) | | | 0.622 | (0.293, 1.321) | 1.183 | (0.658, 2.126) |
| P-value | | | 0.2171 | | 0.5742 | |
| Patient annualized relapse | | | | | | |
| rate^{c} | | | | | | |
| N | 41 | | 42 | | 40 | |
| Mean (SD) | 0.517 | (0.758) | 0.516 | (2.041) | 0.639 | (1.012) |

| | | | |
|---|---|---|---|
| Median | 0.000 | 0.000 | 0.000 |
| Min:Max | 0.00:2.85 | 0.00:13.04 | 0.00:4.35 |

^{a}The total number of confirmed relapses that occurred during the study divided by the total number of patient-years followed in the study.
^{b}Poisson model with the total number of confirmed relapses onset between randomization date and last dose date as the response variable, treatment and region as covariates, and log-transformed standardized study duration as an offset variable.
^{c}The number of confirmed relapses for each patient divided by the number of years followed in the study for that patient.

We claim:

1. A method for reducing the number of T1-Gd lesions in a patient afflicted with relapsing-remitting form of multiple sclerosis comprising administering to the patient about 7 mg of teriflunomide once a day, and a stable dose of glatiramer acetate, wherein the method is clinically proven safe and effective.

2. The method according to claim 1, wherein the stable dose of glatiramer acetate is about 20 mg of glatiramer acetate daily, and is administered concurrently with teriflunomide

3. The method according to claim 2, wherein more number of T1-Gd lesions is reduced in the patient treated by the method than in a patient treated by a stable dose of glatiramer acetate alone, wherein the method is clinically proven effective.

4. A method for reducing the volume of T1-Gd lesions in patients afflicted with relapsing-remitting form of multiple sclerosis, comprising administering to the patients about 14 mg of teriflunomide once a day, and a stable dose of glatiramer acetate, wherein the method is clinically proven safe and effective.

5. The method according to claim 4, wherein the stable dose of glatiramer acetate is about 20 mg of glatiramer acetate daily, and is administered concurrently with teriflunomide.

6. The method according to claim 5, wherein more volume of T1-Gd lesions is reduced in the patient treated by the method than in a patient treated by a stable dose of glatiramer acetate alone.

7. A method for treating relapsing-remitting form of multiple sclerosis in patients in need thereof comprising administering to the patients 14 mg of teriflunomide once a day and a stable dose of glatiramer acetate, wherein about 81.6% of the patients are free of T1-Gd lesions after about 24-week treatment.

8. The method according to claim 7, wherein about 78.9% of the patients are free of T1-Gd lesions after about 48-week treatment.

9. A method for reducing annualized relapse rate in a patient afflicted with relapsing-remitting form of multiple sclerosis, comprising concurrently administering to the patient about 7 mg of teriflunomide once a day, and a stable dose of glatiramer acetate, wherein the patient treated by the method has an annualized relapse rate lower than a patient treated by a stable dose of glatiramer acetate alone.

10. The method according to claim 9, wherein the stable dose of glatiramer acetate is about 20 mg of glatiramer acetate daily, and is administered concurrently with teriflunomide.
